(19) United States
(12) Patent Application Publication
(10) Pub. No.: US 2017/0323069 A1
(43) Pub. Date: Nov. 9, 2017

(54) SYSTEMS AND METHODS FOR MEDICAL INSTRUMENT PATIENT MEASUREMENTS

(71) Applicant: James Stewart Bates, Paradise Valley, AZ (US)

(72) Inventor: James Stewart Bates, Paradise Valley, AZ (US)

(21) Appl. No.: 15/352,488

(22) Filed: Nov. 15, 2016

Related U.S. Application Data
(60) Provisional application No. 62/332,422, filed on May 5, 2016.

(51) Int. Cl. G06F 19/00; G09B 19/00; A61B 5/00

(52) U.S. Cl. CPC G06F 19/3406; A61B 5/6844; G09B 19/003

(57) ABSTRACT

Presented are systems and methods that provide diagnostic measurement tools that enable even laymen to reliably and accurately perform clinical-grade diagnostic measurements of key vital signs with little or no intervention by a health care professional. In various embodiments, this is accomplished by using an automated medical diagnostic system that provides clear and concise audio/video guidance to the patient and monitors the patient's equipment usage to generate high-accuracy measurement data that may be analyzed locally and shared with health care professionals and specialists, as needed.

### CROSS REFERENCE TO RELATED PATENT APPLICATIONS

The present application is a continuation of co-pending U.S. patent application Ser. No. 15/344,390, entitled “SYSTEMS AND METHODS FOR AUTOMATED MEDICAL DIAGNOSTICS” naming as inventor James Stewart Bates, filed Nov. 4, 2016, which claims priority benefit under 35 U.S.C. §119(e), from U.S. Provisional Patent Application No. 62/332,422, entitled “AUTOMATED MEDICAL DIAGNOSTIC SYSTEM,” naming as inventor James Stewart Bates, and filed May 5, 2016, which applications are hereby incorporated herein by reference as to their entire content.

### BACKGROUND

### Technical Field

The present disclosure relates to health care, and more particularly, to self-measurement systems and methods for accurately using medical instruments to perform patient measurements.

### Background of the Invention

Patients' common problems with scheduling an appointment with a primary doctor when needed or in a time-efficient manner is causing a gradual shift away from patients establishing and relying on a life-long relationship with a single general practitioner, who diagnoses and treats a patient in health-related matters, towards patients opting to receive readily available treatment in urgent care facilities that are located near home, work, or school and provide relatively easy access to health care without the inconvenience of appointments that oftentimes must be scheduled weeks or months ahead of time. Yet, the decreasing importance of primary doctors makes it difficult for different treating physicians to maintain a reasonably complete medical record for each patient, which results in a patient having to repeat a great amount of information personal and medical each time when visiting a different facility or different doctor. In some cases, patients confronted with lengthy and time-consuming patient questionnaires fail to provide accurate information that may be important for a proper medical treatment, whether for the sake of expediting their visit or other reasons. In addition, studies have shown that patients attending urgent care or emergency facilities may in fact worsen their health conditions due to the risk of exposure to bacteria or viruses in medical facilities despite the medical profession's efforts to minimize the number of such instances.

Through consistent regulation changes, electronic health record changes and pressure from payers, both health care facilities and providers are looking for ways to make patient intake, triage, diagnosis, treatment, electronic health record data entry, treatment, billing, and patient follow-up activity more efficient, provide better patient experience, and increase the doctor to patient throughput per hour, while simultaneously reducing cost.

The desire to increase access to health care providers, a pressing need to reduce health care costs in developed countries and the goal of making health care available to a larger population in less developed countries have fueled the idea of telemedicine. In most cases, however, video or audio conferencing with a doctor does not provide sufficient patient-physician interaction that is necessary to allow for a proper medical diagnosis to efficiently serve patients.

What is needed are systems and methods that ensure reliable remote or local medical patient intake, triage, diagnosis, treatment, electronic health record data entry/management, treatment, billing and patient follow-up activity so that physicians can allocate patient time more efficiently and, in some instances, allow individuals to manage their own health, thereby, reducing health care costs.

### BRIEF DESCRIPTION OF THE DRAWINGS

References will be made to embodiments of the invention, examples of which may be illustrated in the accompanying figures. These figures are intended to be illustrative, not limiting. Although the invention is generally described in the context of these embodiments, it should be understood that it is not intended to limit the scope of the invention to these particular embodiments.

FIG. 1 illustrates an exemplary diagnostic system according to embodiments of the present disclosure.

FIG. 2 illustrates an exemplary medical instrument equipment system according to embodiments of the present disclosure.

FIG. 3 illustrates an exemplary medical instrument equipment system coupled to a tablet or PC, according to embodiments of the present disclosure.

FIG. 4 illustrates a sensor board comprising an exemplary medical instrument equipment system, according to embodiments of the present disclosure.

FIG. 5 is a flowchart of an illustrative process for making accurate medical instrument patient measurements, according to embodiments of the present disclosure.

FIG. 6 depicts a simplified block diagram of a computing device/information handling system according to embodiments of the present disclosure.

### DETAILED DESCRIPTION OF THE PREFERRED EMBODIMENTS

In the following description, for purposes of explanation, specific details are set forth in order to provide an understanding of the disclosure. It will be apparent, however, to one skilled in the art that the disclosure can be practiced without these details. Furthermore, one skilled in the art will recognize that embodiments of the present disclosure, described below, may be implemented in a variety of ways, such as a process, an apparatus, a system, a device, or a method on a tangible computer-readable medium.

Elements/components shown in diagrams are illustrative of exemplary embodiments of the disclosure and are meant to avoid obscuring the disclosure. It shall also be understood that throughout this discussion that components may be described as separate functional units, which may comprise sub-units, but those skilled in the art will recognize that various components, or portions thereof, may be divided into separate components or may be integrated together, including integrated within a single system or component. It should be noted that functions or operations discussed herein may be implemented as components/elements. Components/elements may be implemented in software, hardware, or a combination thereof.

Furthermore, connections between components or systems within the figures are not intended to be limited to direct connections. Rather, data between these components may be modified, re-formatted, or otherwise changed by intermediary components. Also, additional or fewer connections may be used. Also, additional or fewer connections may be used. It shall also be noted that the terms “coupled” “connected” or “communicatively coupled” shall be understood to include direct connections, indirect connections through one or more intermediary devices, and wireless connections.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” or “in embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims.

Furthermore, the use of certain terms in various places in the specification is for illustration and should not be construed as limiting. A service, function, or resource is not limited to a single service, function, or resource; usage of these terms may refer to a grouping of related services, functions, or resources, which may be distributed or aggregated.

In this document, the term “sensor” refers to a device capable of acquiring information related to any type of physiological condition or activity (e.g., a biometric diagnostic sensor); physical data (e.g., a weight); and environmental information (e.g., ambient temperature sensor), including hardware-specific information. The term “position” refers to spatial and temporal data (e.g., orientation and motion information). “Doctor” refers to any health care professional, health care provider, physician, or person directed by a physician. “Patient” is any user who uses the systems and methods of the present invention, e.g., a person being examined or anyone assisting such person. The term illness may be used interchangeably with the term diagnosis. As used herein, “answer” or “question” refers to one or more of 1) an answer to a question, 2) a measurement or measurement request (e.g., a measurement performed by a “patient”), and 3) a symptom (e.g., a symptom selected by a “patient”).

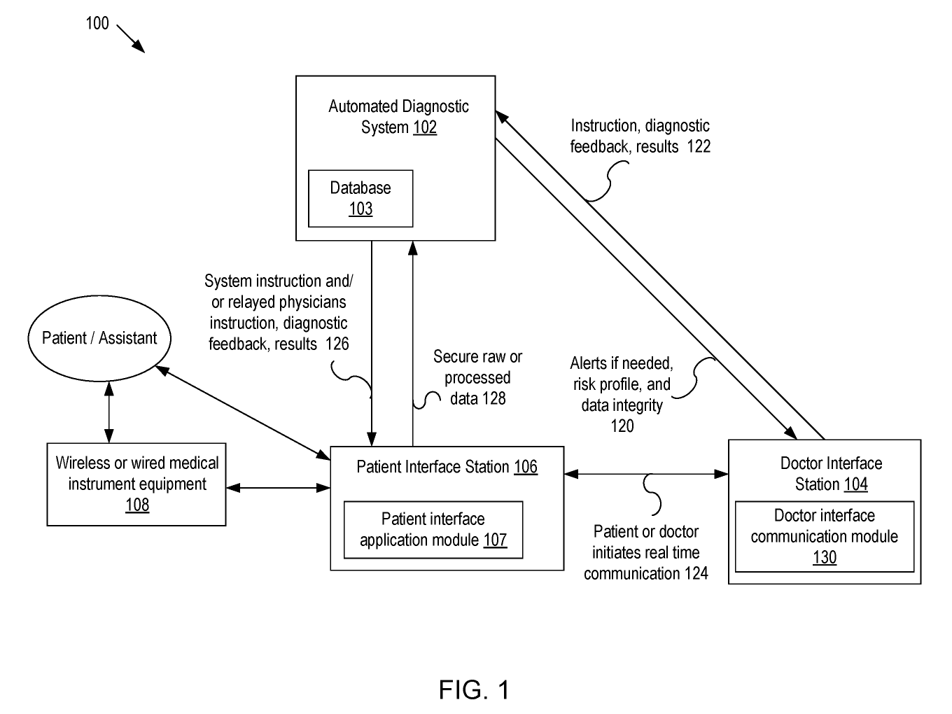

FIG. 1 illustrates an exemplary diagnostic system according to embodiments of the present disclosure. Diagnostic system 100 comprises automated diagnostic system 102, patient interface station 106, doctor interface station 104, and medical instrument equipment 108. Both patient interface station 106 and doctor interface station 104 may be implemented into any tablet, computer, mobile device, or other electronic device. Medical instrument equipment 108 is designed to collect mainly diagnostic patient data, and may comprise one or more diagnostic devices, for example, in a home diagnostic medical kit that generates diagnostic data based on physical and non-physical characteristics of a patient. It is noted that diagnostic system 100 may comprise additional sensors and devices that, in operation, collect, process, or transmit characteristic information about the patient, medical instrument usage, orientation, environmental parameters such as ambient temperature, humidity, location, and other useful information that may be used to accomplish the objectives of the present invention.

In operation, a patient may enter patient-related data, such as health history, patient characteristics, symptoms, health concerns, medical instrument measured diagnostic data, images, and sound patterns, or other relevant information into patient interface station 106. The patient may use any means of communication, such as voice control, to enter data, e.g., in the form of a questionnaire. Patient interface station 106 may provide the data raw or in processed form to automated diagnostic system 102, e.g., via a secure communication.

In embodiments, the patient may be prompted, e.g., by a software application, to answer questions intended to aid in the diagnosis of one or more medical conditions. The software application may provide guidance by describing how to use medical instrument equipment 108 to administer a diagnostic test or how to make diagnostic measurements for any particular device that may be part of medical instrument equipment 108 so as to facilitate accurate measurements of patient diagnostic data.

In embodiments, the patient may use medical instrument equipment 108 to create a patient health profile that serves as a baseline profile. Gathered patient-related data may be securely stored in database 103 or a secure remote server (not shown) coupled to automated diagnostic system 102. In embodiments, automated diagnostic system 102 enables interaction between a patient and a remotely located health care professional, who may provide instructions to the patient, e.g., by communicating via the software application. A doctor may log into a cloud-based system (not shown) to access patient-related data via doctor interface station 104. In embodiments, automated diagnostic system 102 presents automated diagnostic suggestions to a doctor, who may verify or modify the suggested information.

In embodiments, based on one more patient questionnaires, data gathered by medical instrument equipment 108, patient feedback, and historic diagnostic information, the patient may be provided with instructions, feedback, results 122, and other information pertinent to the patient's health. In embodiments, the doctor may select the illness based on the automated diagnostic system suggestions and/or follow a sequence of instructions, feedback, and/or results 122 may be adjusted based on decision vectors associated with a medical database. In embodiments, medical instrument equipment 108 uses the decision vectors to generate a diagnostic result, e.g., in response to patient answers and/or measurements of the patient's vital signs.

In embodiments, medical instrument equipment 108 comprises a number of sensors, such as accelerometers, gyroscopes, pressure sensors, cameras, bolometers, altimeters, IR LEDs, and proximity sensors that may be coupled to one or more medical devices, e.g., a thermometer, to assist in performing diagnostic measurements and/or monitor a patient's use of medical instrument equipment 108 for accuracy. A camera, bolometer, or other spectrum imaging device such as radar, in addition to taking pictures of the patient, may use image or facial recognition software and machine vision to recognize the body parts, items and actions to aid the patient in locating suitable positions for taking a measurement on the patient's body. Facial and body part recognition may serve to identify any part of the patient's body as a reference.

Examples of the types of diagnostic data that medical instrument equipment 108 may generate comprise body temperature, blood pressure, images, sound, heart rate, blood oxygen level, motion, ultrasound, pressure or gas analysis, continuous positive airway pressure, electrocardiogram, electroencephalogram, Electrocardiography, BMI, muscle mass, blood, urine, and any other patient-related data 128. In embodiments, patient-related data 128 may be derived from a non-surgical wearable or implantable monitoring device that gathers sample data.

In embodiments, an IR LED, proximity beacon, or other identifiable marker (not shown) may be affixed to medical instrument equipment 108, e.g., a temperature sensor, to track the position and placement of medical instrument equipment 108. In embodiments, a camera, bolometer, or other spectrum imaging device uses unique markers as a control tool to aid the camera/patient in determining the position of medical instrument equipment 108.

In embodiments, machine vision software may be used to track and overlay or superimpose, e.g., on a screen, the position of the identifiable marker e.g, IR LED, heat source, or reflective material with a desired target location at which the patient should place medical instrument equipment 108, thereby, aiding the patient to properly place or align a sensor and ensure accurate and reliable readings. Once medical instrument equipment 108, e.g., a stethoscope is placed at the desired target location on a patient's torso, the patient may be prompted by optical or visual cues to breath according to instructions or perform other actions to facilitate medical measurements and to start the measurement.

In embodiments, one or more sensors that may be attached to medical instrument equipment 108 monitor the placement and usage of medical instrument equipment 108 by periodically or continuously recording data and comparing measured data, such as location, movement, and angles, to an expected data model and/or an error threshold to ensure measurement accuracy. A patient may be instructed to adjust an angle, location, or motion of medical instrument equipment 108, e.g., to adjust its state and, thus, avoid low-accuracy or faulty measurement readings. Sensors attached or tracking medical instrument equipment 108 and patient interaction activity output may be compared, for example, against an idealized patient medical instrument equipment usage sensor model output creating an accuracy score. The patient medical instrument equipment measured medical data may also be compared with ideal device measurement data expected from medical instrument equipment 108 and compared against a threshold creating an accuracy score. Feedback from medical instrument equipment 108 (e.g., sensors, proximity, camera . . . ) and actual measurement data may be used to instruct the patient to properly align medical instrument equipment 108 during a measurement. In embodiments, medical instrument equipment type and sensor system monitoring of medical instrument equipment 108 patient interaction may be used to create a device usage accuracy score for use in a medical diagnosis algorithm. Similarly, patient medical instrument equipment measured medical data may be used to create a measurement accuracy score for use by the medical diagnostic algorithm.

In embodiments, machine vision software may be used to show animation on the monitor that mimics a patient's movements and provides detailed interactive instructions and real-time feedback to the patient. This aids the patient in correctly positioning and operating medical instrument equipment 108 relative to the patient's body so as to ensure a high level of accuracy when using medical instrument equipment 108.

In embodiments, once automated diagnostic system 102 detects unexpected data, e.g., data representing an unwanted movement, location, measurement data, etc., a validation process comprising a calculation of a trustworthiness score or reliability factor is initiated in order to gauge the measurement accuracy. Once the accuracy of the measured data falls below a desired level, the patient may be asked to either repeat a measurement or request assistance by an assistant, who may answer questions, e.g., remotely via an application to help with proper equipment usage, or alert a nearby person to assist with using medical instrument equipment 108. The validation process, in addition to instructing the patient to repeat a measurement and answer additional questions, may comprise calculating a measurement accuracy score based on a measurement or re-measurement.

In embodiments, upon request 124 automated diagnostic system 102 may enable a patient-doctor interaction by granting the patient and doctor access to diagnostic system 100. The patient may enter data, take measurements, and submit images and audio files or any other information to the application or web portal. The doctor may access that information, for example, to review a diagnosis generated by automated diagnostic system 102, and generate, confirm, or modify instructions for the patient. Patient-doctor interaction, while not required for diagnostic and treatment, if used, may occur in person, real-time via an audio/video application, or by any other means of communication.

In embodiments, automated diagnostic system 102 may utilize images generated from a diagnostic examination of mouth, throat, eyes, ears, skin, extremities, surface abnormalities, internal imaging sources, and other suitable images and/or audio data generated from diagnostic examination of heart, lungs, abdomen, chest, joint motion, voice, and any other audio data sources. Automated diagnostic system 102 may further utilize patient lab tests, medical images, or any other medical data. In embodiments, automated diagnostic system 102 enables medical examination of the patient, for example, using medical devices, e.g., ultrasound, in medical instrument equipment 108 to detect sprains, contusions, or fractures, and automatically provide diagnostic recommendations regarding a medical condition of the patient.

In embodiments, diagnosis comprises medical database decision vectors that are at least partially based on the patient's self-measured (or assistant measured) vitals or other measured medical data. In embodiments, the accuracy score of a measurement dataset, a usage accuracy score of a sensor attached to medical instrument equipment 108, the regional illness trends, and other information used in generally accepted medical knowledge evaluations steps. The decision vectors and associated algorithm, which may be installed in automated diagnostic system 102, may utilize one or more-dimensional data, patient history, patient questionnaire feedback, and pattern recognition or pattern matching for classification using images and audio data. In embodiments, a medical device usage accuracy score generator (not shown) may be implemented within automated diagnostic system 102 and may utilize an error vector of any device in medical instrument equipment or attached sensors 108 to create a device usage accuracy score and utilize the actual patient-measured device data to create a measurement data accuracy score.

In embodiments, automated diagnostic system 102 outputs diagnosis and/or treatment information that is communicated to the patient, for example, by electronically communicating to the patient or through a medical professional either electronically or in person a treatment guideline that may include a prescription for medication. In embodiments, prescriptions may be communicated directly to a pharmacy for pick-up or automated home delivery.

In embodiments, automated diagnostic system 102 may generate an overall health risk profile of the patient and recommend steps to reduce the risk of overlooking potentially dangerous conditions or guide the patient to a nearby facility that can treat the potentially dangerous condition. The health risk profile may assist a treating doctor in fulfilling duties to the patient, for example, to carefully review and evaluate the patient and, if deemed necessary, refer the patient to a specialist, initiate further testing, etc. The health risk profile advantageously reduces the potential for negligence and, thus, medical malpractice lawsuits.

Automated diagnostic system 102, in embodiments, comprises a payment feature that uses patient identification information to access a database to determine if a patient has previously arranged a method of payment. If the patient database does not indicate a previously arranged method of payment, automated diagnostic system 102 may prompt the patient to enter payment information, such as insurance, bank, or credit card information. Automated diagnostic system 102 may determine whether payment information is valid and automatically obtain an authorization from the insurance, EHR system and/or the card issuer for payment for a certain amount for services rendered by the doctor. An invoice may be electronically presented to the patient, e.g., upon completion of a consultation, such that the patient can authorize payment of the invoice, e.g., via an electronic signature.

In embodiments, patient database 103 (e.g., a secured cloud-based database) may comprise a security interface (not shown) that allows secure access to a patient database, for example, by using patient identification information to obtain the patient's medical history. The interface may utilize biometric, bar code, or other electronically security methods. In embodiments, medical instrument equipment 108 uses unique identifiers that are used as a control tool for measurement data. Database 103 may be a repository for any type of data created, modified, or received by diagnostic system 100, such as generated diagnostic information, information received from patient's wearable electronic devices, remote video/audio data and instructions, e.g., instructions received from a remote location or from the application.

In embodiments, fields in the patient's electronic health care record (EHR) are automatically populated based on one or more of questions asked by diagnostic system 100, measurements taken by the patient/system 100, diagnosis and treatment codes generated by system 100, one or more trust scores, and imported patient health care data from one or more sources, such as an existing health care database. It is understood the format of imported patient health care data may be converted to become compatible with the EHR format of system 100. Conversely, exported patient health care data may be converted to be compatible, e.g., with an external EHR database.

In addition, patient-related data documented by system 100 provide support for the code decision for the level of exam a doctor performs. As in existing methods, doctors have to choose, for billing and reimbursement purposes, one of any identified codes (e.g., ICD10 currently holds approximately 97,000 medical codes) to identify an illness and provide an additional code that identifies the level of physical exam/diagnosis performed on the patient (e.g., full body physical exam) based on the illness identified by the doctor.

In embodiments, the documented questions are used to suggest to the doctor a level of exam that is supported by the illness identified so as to ensure that, e.g., the doctor does not perform unnecessary in-depth exams for minor illnesses or performs treatment that may not be covered by the patient's insurance.

In embodiments, upon identifying a diagnosis, system 100 generates one or more recommendations/suggestions/options for a particular treatment. In embodiments, one or more treatment plans are generated that the doctor may discuss with the patient and decide on a suitable treatment. For example, one treatment plan may be tailored purely for effectiveness, another treatment plan may consider drug costs. In embodiments, system 100 may generate a prescription/lab test request and considers factors, such as recent research results, available drugs and possible drug interactions, the patient's medical history, traits of the patient, family history and any other factors that may affect treatment to provide treatment information for a doctor. In embodiments, diagnosis and treatment databases may be continuously updated, e.g., by health care professionals, so that an optimal treatment for a particular patient, e.g., a patient identified as member of a certain risk group, may be administered.

It is noted that sensors and measurement techniques may be advantageously combined to perform multiple functions using a reduced number of sensors. For example, an optical sensor may be used as a thermal sensor by utilizing IR technology to measure body temperature. It is further noted that some or all data collected by system 100 may be processed and analyzed directly within automated diagnostic system 102 or transmitted to an external reading device (not shown in FIG. 1) for further processing and analysis, e.g., to enable additional diagnostics.

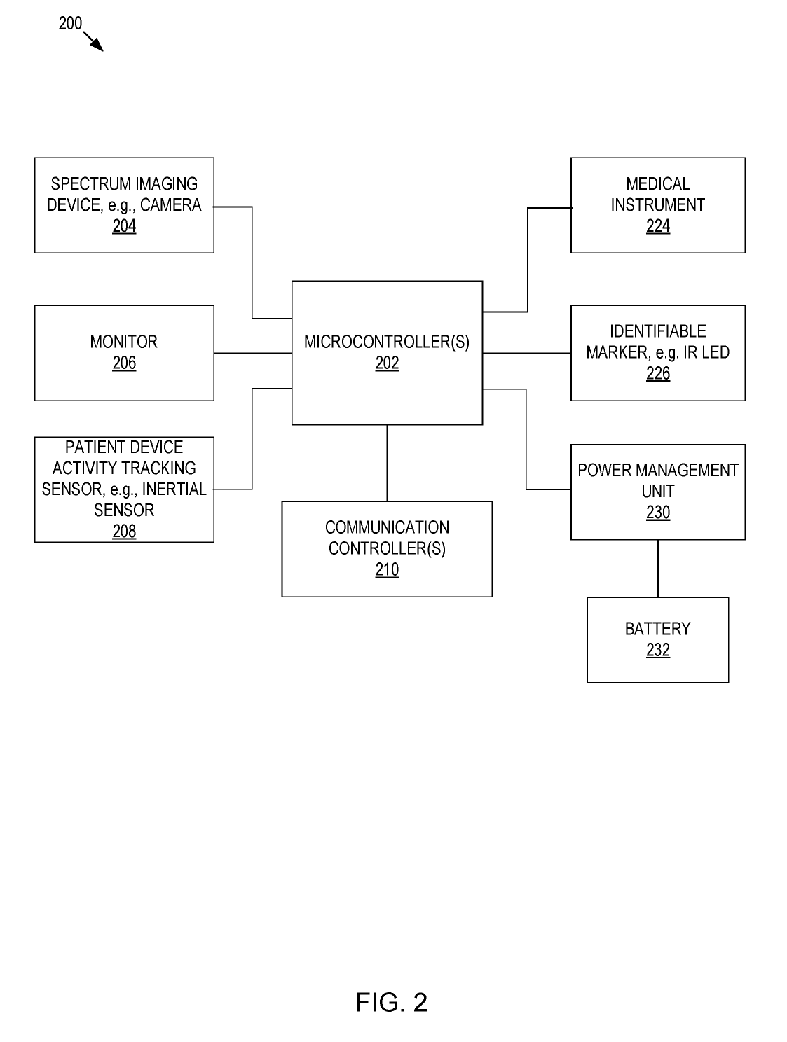

FIG. 2 illustrates an exemplary patient diagnostic measurement system according to embodiments of the present disclosure. As depicted, patient diagnostic measurement system 200 comprises microcontroller 202, spectrum imaging device, e.g., camera 204, monitor 206, patient-medical equipment activity tracking sensors, e.g., inertial sensor 208, communications controller 210, medical instruments 224, identifiable marker, e.g., IR LED 226, power management unit 230, and battery 232. Each component may be coupled directly or indirectly by electrical wiring, wirelessly, or optically to any other component in system 200.

Medical instrument 224 comprises one or more devices that are capable of measuring physical and non-physical characteristics of a patient that, in embodiments, may be customized, e.g., according to varying anatomies among patients, irregularities on a patient's skin, and the like. In embodiments, medical instrument 224 is a combination of diagnostic medical devices that generate diagnostic data based on patient characteristics. Possible diagnostic medical devices are, for example, heart rate sensor, otoscope, digital stethoscope, in-ear thermometer, blood oxygen sensor, high-definition camera, spirometer, blood pressure meter, respiration sensor, skin resistance sensor, glucometer, ultrasound, electrocardiographic sensor, body fluid sample collector, eye slit lamp, weight scale, and any other device known in the art that may aid in performing a medical diagnosis. In embodiments, patient characteristics and vital signs data may be received from and/or compared against wearable or implantable monitoring devices that gather sample data, e.g., a fitness device that monitors physical activity.

One or more medical instruments 224 may removably attachable directly to a patient's body, e.g., the patient's torso, via patches or electrodes that may use adhesion to provide good physical or electrical contact. In embodiments, medical instruments 224, such as a contact-less thermometer, may perform contact-less measurements some distance away from the patient's body.

In embodiments, microcontroller 202 may be a secure microcontroller that securely communicates information in encrypted form to ensure privacy and the authenticity of measured data and activity sensor and patient-equipment proximity information and other information in patient diagnostic measurement system 200. This may be accomplished by taking advantage of security features embedded in hardware of microcontroller 202 and/or software that enables security features during transit and storage of sensitive data. Each device in patient diagnostic measurement system 200 may have keys that handshake to perform authentication operations on a regular basis.

Spectrum imaging device camera 204 is any audio/video device that may capture patient images and sound at any frequency or image type. Monitor 206 is any screen or display device that may be coupled to camera, sensors and/or any part of system 200. Patient-equipment activity tracking inertial sensor 208 is any single or multi-dimensional sensor, such as an accelerometer, a multi-axis gyroscope, pressure, and a magnetometer capable of providing position, motion, pressure on medical equipment or orientation data. Patient-equipment activity tracking inertial sensor 208 may be attached to (removably or permanently) or embedded into medical instrument 224. Identifiable marker IR LED 226 represents any device, heat source, reflective material, proximity beacon, altimeter, etc., that may be used by microcontroller 202 as an identifiable marker. Like patient-equipment activity tracking inertial sensor 208, identifiable marker IR LED 226 may be reattached to or embedded into medical instrument 224.

In embodiments, communication controller 210 is a wireless communications controller attached either permanently or temporarily to medical instrument 224 or the patient's body to establish a bi-directional wireless communications link and transmit data, e.g., between sensors and microcontroller 202 using any wireless communication protocol known in the art, such as Bluetooth Low Energy, e.g., via an embedded antenna circuit that wirelessly communicates the data. One of ordinary skill in the art will appreciate that electromagnetic fields generated by such antenna circuit may be of any suitable type. In case of an RF field, the operating frequency may be located in the ISM frequency band, e.g., 13.56 MHz. In embodiments, data received by wireless communications controller 210 may be forwarded to a host device (not shown) that may run a software application.

In embodiments, power management unit 230 is coupled to microcontroller 202 to provide energy to, e.g., microcontroller 202 and communication controller 210. Battery 232 may be a back-up battery for power management unit 230 or a battery in any one of the devices in patient diagnostic measurement system 200. One of ordinary skill in the art will appreciate that one or more devices in system 200 may be operated from the same power source (e.g., battery 232) and perform more than one function at the same or different times. A person of skill in the art will also appreciate that one or more components, e.g., sensors 208, 226, may be integrated on a single chip/system, and that additional electronics, such as filtering elements, etc., may be implemented to support the functions of medical instrument equipment measurement or usage monitoring and tracking system 200 according to the objectives of the invention.

In operation, a patient may use medical instrument 224 to gather patient data based on physical and non-physical patient characteristics, e.g., vital signs data, images, sounds, and other information useful in the monitoring and diagnosis of a health-related condition. The patient data is processed by microcontroller 202 and may be stored in a database (not shown). In embodiments, the patient data may be used to establish baseline data for a patient health profile against which subsequent patient data may be compared.

In embodiments, patient data may be used to create, modify, or update EHR data. Gathered medical instrument equipment data, along with any other patient and sensor data, may be processed directly by patient diagnostic measurement system 200 or communicated to a remote location for analysis, e.g., to diagnose existing and expected health conditions to benefit from early detection and prevention of acute conditions or aid in the development of novel medical diagnostic methods.

In embodiments, medical instrument 224 is coupled to a number of sensors, such as patient-equipment tracking inertial sensor 208 and/or identifiable marker IR LED 226, that may monitor a position/orientation of medical instrument 224 relative to the patient's body when a medical equipment measurement is taken. In embodiments, sensor data generated by sensor 208, 226 or other sensors may be used in connection with, e.g., data generated by spectrum imaging device camera 204, proximity sensors, transmitters, bolometers, or receivers to provide feedback to the patient to aid the patient in properly aligning medical instrument 224 relative to the patient's body part of interest when performing a diagnostic measurement. A person skilled in the art will appreciate that not all sensors 208, 226, beacon, pressure, altimeter, etc., need to operate at all times. Any number of sensors may be partially or completely disabled, e.g., to conserve energy.

In embodiments, the sensor emitter comprises a light signal emitted by IR LED 226 or any other identifiable marker that may be used as a reference signal. In embodiments, the reference signal may be used to identify a location, e.g., within an image and based on a characteristic that distinguishes the reference from other parts of the image. In embodiments, the reference signal is representative of a difference between the position of medical instrument 224 and a preferred location relative to a patient's body. In embodiments, spectrum imaging device camera 204 displays, e.g., via monitor 206, the position of medical instrument 224 and the reference signal at the preferred location so as to allow the patient to determine the position of medical instrument 224 and adjust the position relative to the preferred location, displayed by spectrum imaging device camera 204.

Spectrum imaging device camera 204, proximity sensor, transmitter, receiver, bolometer, or any other suitable device may be used to locate or track the reference signal, e.g., within the image, relative to a body part of the patient. In embodiments, this may be accomplished by using an overlay method that overlays an image of a body part of the patient against an ideal model of device usage to enable real-time feedback for the patient. The reference signal along with signals from other sensors, e.g., patient-equipment activity inertial sensor 208, may be used to identify a position, location, angle, orientation, or usage associated with medical instrument 224 to monitor and guide a patient's placement of medical instrument 224 at a target location and accurately activate a device for measurement.

In embodiments, e.g., upon receipt of a request signal, microcontroller 202 activates one or more medical instruments 224 to perform measurements and sends data related to the measurement back to microcontroller 202. The measured data and other data associated with a physical condition may be automatically recorded and a usage accuracy of medical instrument 224 may be monitored.

In embodiments, microcontroller 202 uses an image in any spectrum, motion signal and/or an orientation signal by patient-equipment activity inertial sensor 208 to compensate or correct the vital signs data output by medical instrument 224. Data compensation or correction may comprise filtering out certain data as likely being corrupted by parasitic effects and erroneous readings that result from medical instrument 224 being exposed to unwanted movements caused by perturbations or, e.g., the effect of movements of the patient's target measurement body part.

In embodiments, signals from two or more medical instruments 224, or from medical instrument 224 and patient-activity activity system inertial sensor 208, are combined, for example, to reduce signal latency and increase correlation between signals to further improve the ability of vital signs measurement system 200 to reject motion artifacts to remove false readings and, therefore, enable a more accurate interpretation of the measured vital signs data.

In embodiments, spectrum imaging device camera 204 displays actual or simulated images and videos of the patient and medical instrument 224 to assist the patient in locating a desired position for medical instrument 224 when performing the measurement so as to increase measurement accuracy. Spectrum imaging device camera 204 may use image or facial recognition software to identify and display eyes, mouth, nose, ears, torso, or any other part of the patient's body as reference.

In embodiments, vital signs measurement system 200 uses machine vision software that analyzes measured image data and compares image features to features in a database, e.g., to detect an incomplete image for a target body part, to monitor the accuracy of a measurement and determine a corresponding score. In embodiments, if the score falls below a certain threshold system 200 may provide detailed guidance for improving measurement accuracy, e.g., by changing an angle or depth of an otoscope relative to the patient's ear to receive a more complete image.

In embodiments, the machine vision software may use an overlay method to mimic a patient's posture/movements to provide detailed and interactive instructions, e.g., by displaying a character, image of the patient, graphic, or avatar on monitor 206 to provide feedback to the patient. The instructions, image, or avatar may start or stop and decide what help instruction to display based on the type of medical instrument 224, the data from spectrum imaging device camera 204, patient-equipment activity sensors inertial sensors 208, bolometer, transmitter and receiver, and/or identifiable marker IR LED 226 (an image, a measured position or angle, etc.), and a comparison of the data to idealized data. This further aids the patient in correctly positioning and operating medical instrument 224 relative to the patient's body, ensures a high level of accuracy when operating medical instrument 224, and solves potential issues that the patient may encounter when using medical instrument 224.

In embodiments, instructions may be provided via monitor 206 and describe in audio/visual format and in any desired level of detail, how to use medical instrument 224 to perform a diagnostic test or measurement, e.g., how to take temperature, so as to enable patients to perform measurements of clinical grade accuracy. In embodiments, each sensor 208, 226, e.g., proximity, bolometer, transmitter/receiver may be associated with a device usage accuracy score. A device usage accuracy score generator (not shown), which may be implemented in microcontroller 202, may use the sensor data to generate a medical instrument usage accuracy score that is representative of the reliability of medical instrument 224 measurement on the patient. In embodiments, the score may be based on a difference between an actual position of medical instrument 224 and a preferred position. In addition, the score may be based on detecting a motion, e.g., during a measurement. In embodiments, in response to determining that the accuracy score falls below a threshold, a repeat measurement or device usage assistance may be requested. In embodiments, the device usage accuracy score is derived from an error vector generated for one or more sensors 208, 226. The resulting device usage accuracy score may be used when generating or evaluating medical diagnosis data.

In embodiments, microcontroller 202 analyzes the patient measured medical instrument data to generate a trust score indicative of the acceptable range of the medical instrument. For example, by comparing the medical instrument measurement data against reference measurement data or reference measurement data that would be expected from medical instrument 224. As with device usage accuracy score, the trust score may be used when generating or evaluating a medical diagnosis data.

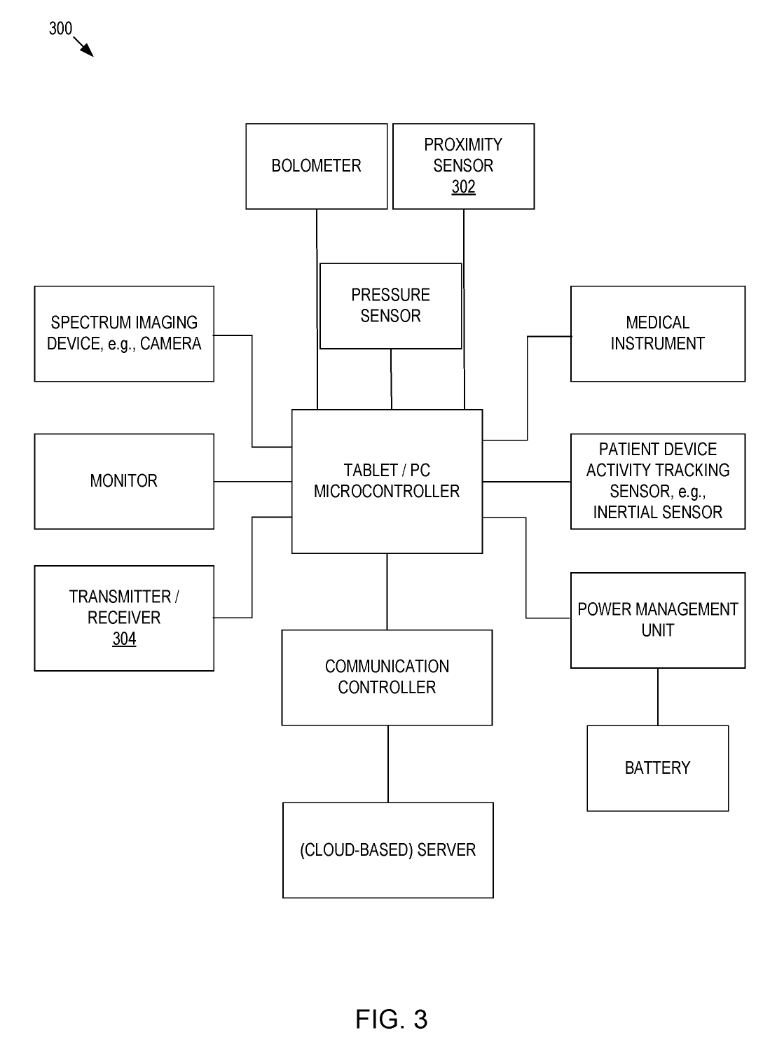

FIG. 3 illustrates an exemplary medical equipment measurement system coupled to a tablet or PC, according to embodiments of the present disclosure. In embodiments, proximity sensor 302 is any device capable of determining a proximity of medical instrument 224 to the patient. Transmitter/Receiver 304 may be any device capable of sending and/or receiving a signal from a beacon measuring proximity or location, or any device (e.g., radar) that takes or receives images of the patient and medical instrument 224 that are outside of the spectrum of camera 204.

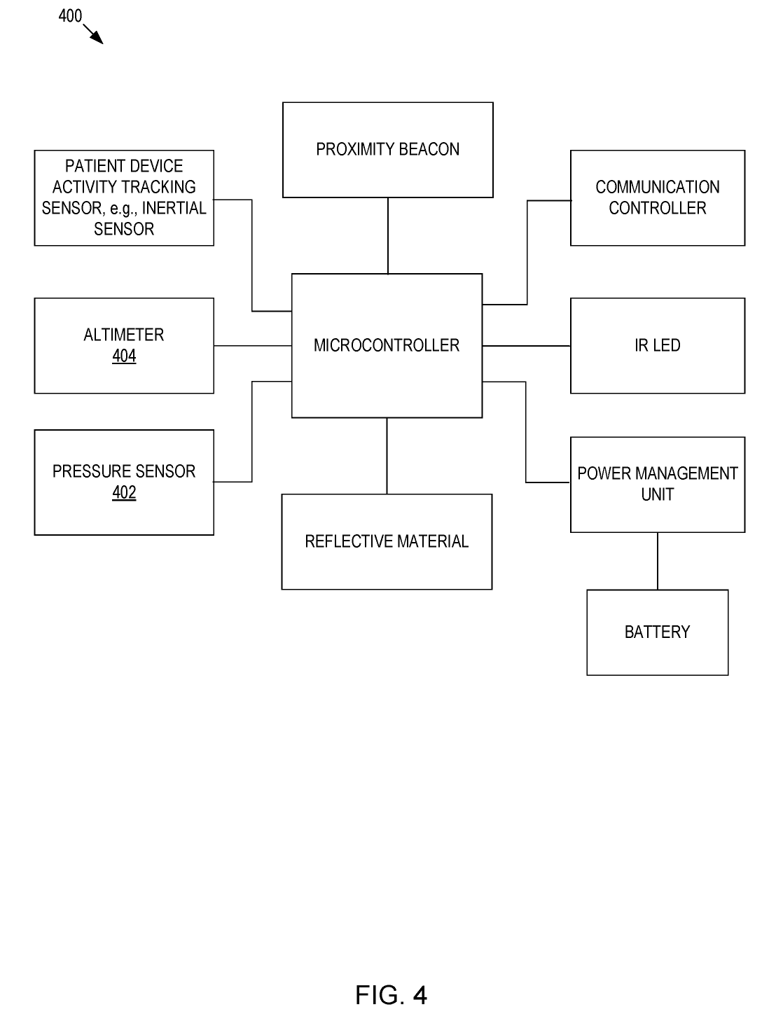

FIG. 4 illustrates a sensor board 400 comprising an exemplary medical instrument equipment system, according to embodiments of the present disclosure. Pressure sensor 402 may be any sensor capable of measuring the pressure on the medical device based on patient interaction, e.g., by a pressure on a handle or a thermometer. In embodiments, altimeter 404 measures an altitude of medical instrument or patient movement.

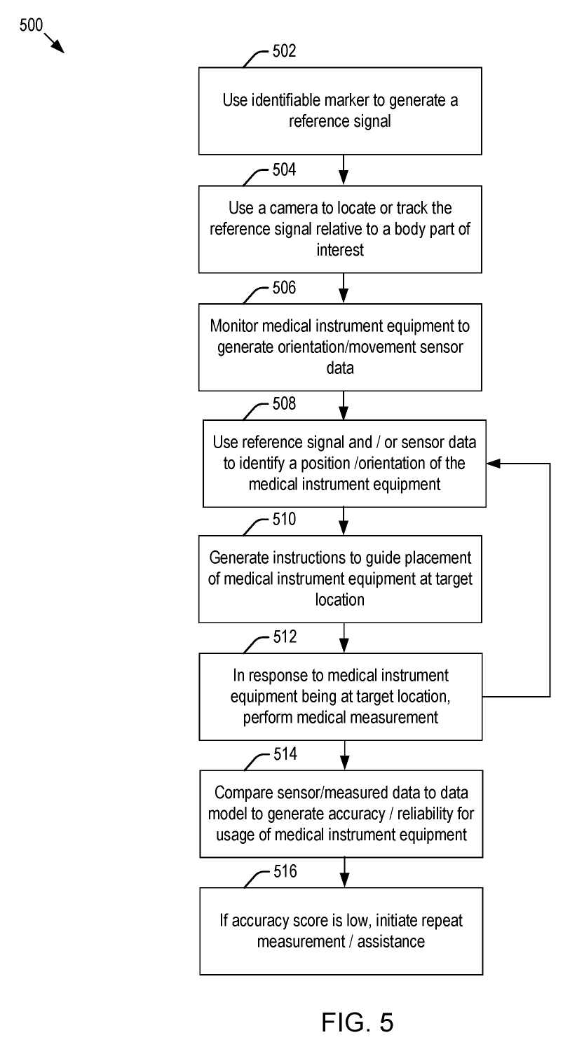

FIG. 5 is a flowchart of an illustrative process for making accurate medical instrument patient measurements, according to embodiments of the present disclosure. Process 500 for accurate measurements starts at step 502 when, for example, in response to a motion detector sensing an acceleration, an identifiable marker, such as an IR LED, heat source, RF beacon, or reflective material is used to generate a reference signal, for example, within an image.

At step 504, the reference signal is located or tracked by a spectrum imaging device, e.g., via a camera, relative to a target location associated with a body part of interest, which may be tracked using machine vision and a spectrum camera or sensor.

At step 506, one or more sensors associated with the medical instrument equipment monitor orientation and/or movement of the medical instrument equipment relative to the patient to generate sensor data, such as initial sensor data.

At step 508, the reference signal is used, e.g., in connection with the sensor data, to identify and monitor a position or orientation associated with a medical instrument relative to the patient.

At step 510, based on the relative position, one or more instructions are generated to guide the patient in properly placing the medical instrument at the target location. For example, when a pressure sensor is used to sense an amount of force exerted on the medical instrument, instructions on the amount of force may be provided.

At step 512, in response to determining that the medical instrument is placed at the target location, the medical instrument equipment may perform vital signs measurements that are automatically recorded. It is understood that if the medical instrument is not used in a proper manner, not placed at the target location, or produces a faulty reading, the patient is instructed to follow instructions to correct the placement or measurement with the medical instrument.

At step 514, the sensor and/or measurement data is compared to a data model and, based on the comparison, an accuracy or reliability score for usage of the medical instrument equipment is assigned to the measurement data. In embodiments, a correction may be applied to the measurement data, e.g., based on a correlation between two or more signals, a filtering process, or a known systematic error.

At step 516, if the score falls below a threshold, a repeat measurement or assistance may be requested. One skilled in the art will recognize that: (1) certain steps may optionally be performed; (2) steps may not be limited to the specific order set forth herein; and (3) certain steps may be performed in different orders; and (4) certain steps may be done concurrently.

In embodiments, one or more computing systems, such as mobile/tablet/computer or the automated diagnostic system, may be configured to perform one or more of the methods, functions, and/or operations presented herein. Systems that implement at least one or more of the methods, functions, and/or operations described herein may comprise an application or applications operating on at least one computing system. The computing system may comprise one or more computers and one or more databases. The computer system may be a single system, a distributed system, a cloud-based computer system, or a combination thereof.

It shall be noted that the present disclosure may be implemented in any instruction-execution/computing device or system capable of processing data, including, without limitation phones, laptop computers, desktop computers, and servers. The present disclosure may also be implemented into other computing devices and systems. Furthermore, aspects of the present disclosure may be implemented in a wide variety of ways including software (including firmware), hardware, or combinations thereof. For example, the functions to practice various aspects of the present disclosure may be performed by components that are implemented in a wide variety of ways including discrete logic components, one or more application specific integrated circuits (ASICs), and/or program-controlled processors. It shall be noted that the manner in which these items are implemented is not critical to the present disclosure.

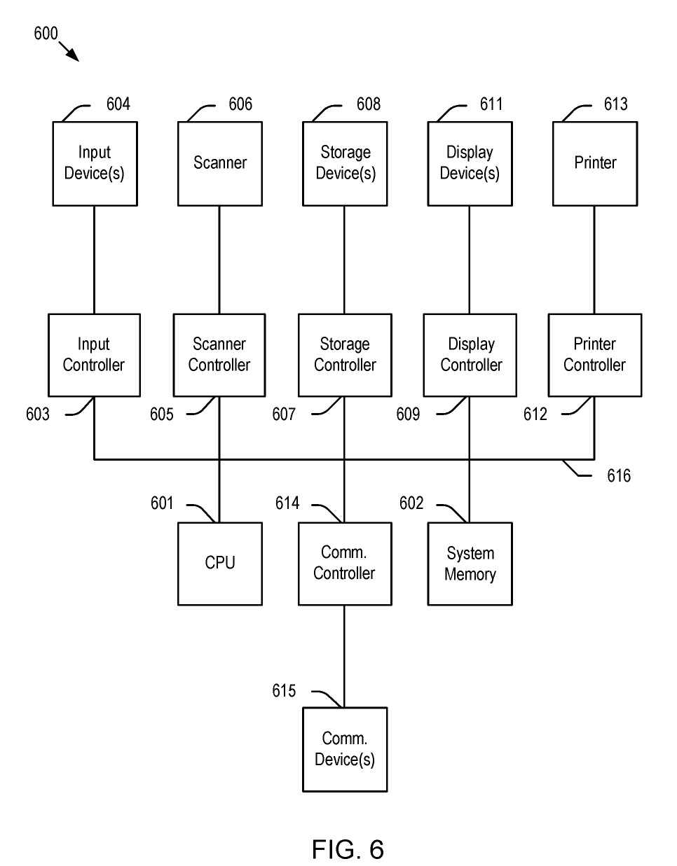

Having described the details of the disclosure, an exemplary system that may be used to implement one or more aspects of the present disclosure is described next with reference to FIG. 6. Each of patient interface station 106 and automated diagnostic system 102 in FIG. 1 may comprise one or more components in the system 600. As illustrated in FIG. 6, system 600 includes a central processing unit (CPU) 601 that provides computing resources and controls the computer. CPU 601 may be implemented with a microprocessor or the like, and may also include a graphics processor and/or a floating point coprocessor for mathematical computations. System 600 may also include a system memory 602, which may be in the form of random-access memory (RAM) and read-only memory (ROM).

A number of controllers and peripheral devices may also be provided, as shown in FIG. 6. An input controller 603 represents an interface to various input device(s) 604, such as a keyboard, mouse, or stylus. There may also be a scanner controller 605, which communicates with a scanner 606. System 600 may also include a storage controller 607 for interfacing with one or more storage devices 608 each of which includes a storage medium such as magnetic tape or disk, or an optical medium that might be used to record programs of instructions for operating systems, utilities and applications which may include embodiments of programs that implement various aspects of the present disclosure. Storage device(s) 608 may also be used to store processed data or data to be processed in accordance with the disclosure. System 600 may also include a display controller 609 for providing an interface to a display device 611, which may be a cathode ray tube (CRT), a thin film transistor (TFT) display, or other type of display. System 600 may also include a printer controller 612 for communicating with a printer 66. A communications controller 614 may interface with one or more communication devices 615, which enables system 600 to connect to remote devices through any of a variety of networks including the Internet, an Ethernet cloud, an FCoE/DCB cloud, a local area network (LAN), a wide area network (WAN), a storage area network (SAN) or through any suitable electromagnetic carrier signals including infrared signals.

In the illustrated system, all major system components may connect to a bus 616, which may represent more than one physical bus. However, various system components may or may not be in physical proximity to one another. For example, input data and/or output data may be remotely transmitted from one physical location to another. In addition, programs that implement various aspects of this disclosure may be accessed from a remote location (e.g., a server) over a network. Such data and/or programs may be conveyed through any of a variety of machine-readable medium including, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices.

Embodiments of the present disclosure may be encoded upon one or more non-transitory computer-readable media with instructions for one or more processors or processing units to cause steps to be performed. It shall be noted that the one or more non-transitory computer-readable media shall include volatile and non-volatile memory. It shall be noted that alternative implementations are possible, including a hardware implementation or a software/hardware implementation. Hardware-implemented functions may be realized using ASIC(s), programmable arrays, digital signal processing circuitry, or the like. Accordingly, the “means” terms in any claims are intended to cover both software and hardware implementations. Similarly, the term “computer-readable medium or media” as used herein includes software and/or hardware having a program of instructions embodied thereon, or a combination thereof. With these implementation alternatives in mind, it is to be understood that the figures and accompanying description provide the functional information one skilled in the art would require to write program code (i.e., software) and/or to fabricate circuits (i.e., hardware) to perform the processing required.

It shall be noted that embodiments of the present disclosure may further relate to computer products with a non-transitory, tangible computer-readable medium that have computer code thereon for performing various computer-implemented operations. The media and computer code may be those specially designed and constructed for the purposes of the present disclosure, or they may be of the kind known or available to those having skill in the relevant arts. Examples of tangible computer-readable media include, but are not limited to: magnetic media such as hard disks, floppy disks, and magnetic tape; optical media such as CD-ROMs and holographic devices; magneto-optical media; and hardware devices that are specially configured to store or to store and execute program code, such as application specific integrated circuits (ASICs), programmable logic devices (PLDs), flash memory devices, and ROM and RAM devices. Examples of computer code include machine code, such as produced by a compiler, and files containing higher level code that are executed by a computer using an interpreter. Embodiments of the present disclosure may be implemented in whole or in part as machine-executable instructions that may be in program modules that are executed by a processing device. Examples of program modules include libraries, programs, routines, objects, components, and data structures. In distributed computing environments, program modules may be physically located in settings that are local, remote, or both.

For purposes of this disclosure, an information handling system may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, an information handling system may be a personal computer (e.g., desktop or laptop), tablet computer, mobile device (e.g., personal digital assistant (PDA) or smart phone), server (e.g., blade server or rack server), a network storage device, or any other suitable device and may vary in size, shape, performance, functionality, and price. The information handling system may include random access memory (RAM), one or more processing resources such as a central processing unit (CPU) or hardware or software control logic, ROM, and/or other types of nonvolatile memory. Additional components of the information handling system may include one or more disk drives, one or more network ports for communicating with external devices as well as various input and output (I/O) devices, such as a keyboard, a mouse, touchscreen and/or a video display. The information handling system may also include one or more buses operable to transmit communications between the various hardware components.

One skilled in the art will recognize no computing system or programming language is critical to the practice of the present disclosure. One skilled in the art will also recognize that a number of the elements described above may be physically and/or functionally separated into sub-modules or combined together.

It will be appreciated to those skilled in the art that the preceding examples and embodiment are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure.

1. A medical data system to generate accurate medical instrument measured data associated with a physical condition comprising:
one or more processors coupled to receive medical instrument measured data from a medical instrument that has a position;
a reference signal indicative of at least one of the position and a preferred location relative to a user's body; and
a feedback device coupled to the one or more processors, the feedback device uses a monitor to display at least one of the reference signal and a feedback signal that is representative of a difference between the position and the preferred location to allow a user to adjust the position relative to the preferred location and, thereby, increase data accuracy when the medical instrument is operated.

2. The medical data system according to claim 1, wherein the reference signal is an identifiable marker.

3. The medical data system according to claim 2, wherein the feedback device comprises a camera that displays the identifiable marker on the monitor.

4. The medical data system according to claim 2, wherein the feedback device comprises a bolometer that generates the identifiable marker.

5. The medical data system according to claim 2, wherein the feedback device comprises one of an RF transmitter and an RF receiver that generates the identifiable marker.

6. The medical data system according to claim 1, wherein the reference signal is generated by an IR LED device.

7. The medical data system according to claim 1, wherein the reference signal is a heat signal that is generated by a heat source.

8. The medical data system according to claim 2, wherein the identifiable marker is generated by an RF reflective material that can be displayed on an RF imaging device.

9. The medical data system according to claim 1, wherein the reference signal is an RF beacon.

10. The medical data system according to claim 1, wherein the reference signal generated by a device that is removably attached to the medical instrument.

11. The medical data system according to claim 1, further comprising an inertial sensor coupled to the medical instrument, the inertial sensor outputs at least one of a motion signal and an orientation signal associated with the medical instrument.

12. The medical data system according to claim 11, wherein the inertial sensor is embedded into the medical instrument.

13. The medical data system according to claim 11, wherein the one or more processors combine at least one of the motion signal and the orientation signal with a medical instrument signal to improve sensor accuracy.

14. The medical data system according to claim 11, wherein the one or more processors combine at least one of the motion signal and the orientation signal with the medical instrument measured data to reduce motion artifacts.

15. The medical data system according to claim 1, further comprising a pressure sensor that, in response to detecting a pressure associated with the medical instrument, generates a pressure reading.

16. The medical data system according to claim 1, further comprising an altimeter that generates altitude data.

17. A medical diagnosis system to generate accurate medical instrument measured data associated with a physical condition comprising:
one or more processors coupled to receive measured data from a medical instrument that has a position;
an identifiable marker that generates a reference signal indicative of at least one of the position and a preferred location relative to a user's body; and
a feedback device coupled to the one or more processors, the feedback device uses a monitor to display at least one of the reference signal and a feedback signal that is representative of a difference between the position and the preferred location to allow a user to adjust the position relative to the preferred location and, thereby, increase data accuracy;
a communication controller coupled to the medical instrument to establish a wireless communication between the medical instrument and the one or more processors; and
power management unit coupled to the one or more processors to provide energy to the communication controller and the one or more processors.

18. The medical diagnosis system according to claim 17, wherein the monitor displays instructions related to using the medical instrument.

19. The medical diagnosis system according to claim 17, wherein the feedback device comprises one of a bolometer, an RF transmitter, and an RF receiver to generate the identifiable marker.

20. The medical diagnosis system according to claim 17, further comprising a proximity sensor that that determines determining a proximity of medical instrument to the user's body.

21. The medical diagnosis system according to claim 17, wherein the medical instrument is a wearable device.

22. The medical diagnosis system according to claim 17, wherein the one or more processors generate an alert in response to the position relative to the preferred location exceeding a predetermined threshold.

23. The medical diagnosis system according to claim 17, wherein, in response to determining that the medical instrument is at the preferred location, one or more processors cause the monitor to display instructions to start a measurement.

24. A method for generating accurate medical instrument measured data associated with a physical condition, the method comprising:
monitoring a target location associated with a body part of interest;
using an identifiable marker to generate a reference signal;
determining a relationship between the reference and the target location;
associating inertial sensor data with a medical instrument;
using the inertial sensor data and the relationship to identify a relative position of the medical instrument;
based on the relative position, generating one or more instructions to guide a user in using the reference signal to place the medical instrument at the target location;
in response to determining that the medical instrument is at the target location, receiving medical instrument measured data by the medical instrument;
comparing at least one of the sensor data and the medical instrument measured data to model data to generate a comparison result;
based on the comparison result, assigning at least one of an accuracy score to the medical instrument and a reliability score to the medical instrument measured data; and
if one of the accuracy and reliability score falls below a threshold generating a request signal.

25. The method according to claim 24, wherein the model data is associated with expected measurement data.

26. The method according to claim 24, wherein the identifiable marker is comprised in an audio file.

27. The method according to claim 24, wherein the identifiable marker is outside the visible spectrum.

28. The method according to claim 24, further comprising superimposing, on a monitor, a representation of a user against a user model and mimicking movements of the user to provide feedback to the user in real-time.

29. The method according to claim 24, further comprising, based on the relationship, generating an error vector for one or more sensors to calculate a device usage accuracy score.
